(19) United States
(12) Patent Application Publication
(10) Pub. No.: US 2019/0125484 A1
(43) Pub. Date: May 2, 2019

(54) ISOMARK TATTOOING DEVICES

(71) Applicant: Mayo Foundation for Medical Education and Research, Rochester, MN (US)

(72) Inventor: Broc T. Giffey, Rochester, MN (US)

(21) Appl. No.: 16/170,613

(22) Filed: Oct. 25, 2018

Related U.S. Application Data
(60) Provisional application No. 62/580,774, filed on Nov. 2, 2017.

(51) Int. Cl. A61B 90/00; A61M 37/00

(52) U.S. Cl. CPC A61B 90/39; A61B 2090/3987; A61B 2090/3933; A61M 37/0084

(57) ABSTRACT

An isocenter marker for marking a treatment isocenter on a patient's body. Embodiments include a base, alignment indicia configured for alignment with external alignment sources to facilitate alignment of the base over an isocenter on a patient's body, an ink reservoir in the base, an ink channel in the base, and an actuator assembly attachable to the base. The actuator assembly includes a rupture member to open the ink reservoir and allow ink from the reservoir to flow to the ink channel when the actuator assembly is attached to the base, an ink marker including a needle with a tip configured for movement through the ink channel and positioned relative to the alignment indicia for marking an ink mark indicating the isocenter, and a marker actuator for actuating the ink marker to drive the needle from a retracted position through the ink channel to a marking position.

### CROSS REFERENCE TO RELATED APPLICATION

This application claims the benefit of U.S. Provisional Application No. 62/580,774 filed on Nov. 2, 2017 and entitled Isomark Tattooing Devices, which is incorporated herein by reference in its entirety and for all purposes.

### FIELD OF THE INVENTION

Embodiments of the invention include devices for marking a treatment isocenter on a patient's body.

### BACKGROUND

Devices for marking a patient's body, and in particular devices for marking or tattooing treatment isocenters, are disclosed in the Ahmann et al. U.S. Pat. No. 9,113,956 and the Giffey U.S. Pat. No. 9,271,805. Both of these patents are incorporated herein by reference and for all purposes. There remains a continuing need for improved isocenter marking devices.

### SUMMARY

Embodiments of an isocenter marker in accordance with the invention include a base, alignment indicia configured for alignment with external alignment sources to facilitate alignment of the base over an isocenter on a patient's body, an ink reservoir in the base, an ink channel in the base, and an actuator assembly attachable to the base. The actuator assembly includes a rupture member to open the ink reservoir and allow ink from the reservoir to flow to the ink channel when the actuator assembly is attached to the base, an ink marker including a needle with a tip configured for movement through the ink channel and positioned relative to the alignment indicia for marking an ink mark indicating the isocenter, and a marker actuator for actuating the ink marker to drive the needle from a retracted position through the ink channel to a marking position. The ink reservoir is attachable to the base in embodiments. In yet other embodiments the ink reservoir is located adjacent to the ink channel.

### BRIEF DESCRIPTION OF THE DRAWINGS

FIG. 1 is a diagrammatic illustration of an isocenter marker in accordance with embodiments.

FIG. 2 is a diagrammatic illustration of an isocenter marker in accordance with embodiments.

FIG. 3 is a diagrammatic illustration of an isocenter marker in accordance with embodiments.

### DETAILED DESCRIPTION

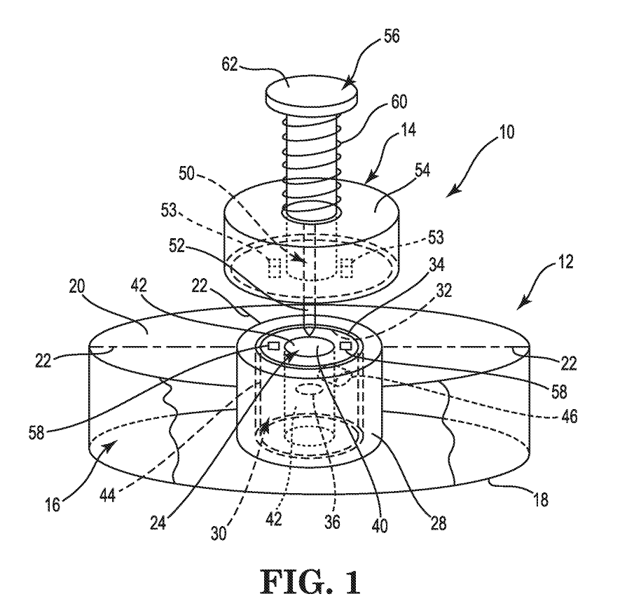

FIG. 1 is a diagrammatic illustration of an isocenter marker 10 in accordance with embodiments of the invention. As shown, marker 10 includes a base assembly 12 and an actuator assembly 14 that can be attached to the base assembly. In the illustrated embodiment the marker 10 is manufactured as a multi-piece device, and the actuator assembly 14 is a separate element that can be mounted to the base assembly 12 during use of the marker 10. In other embodiments the marker 10 is a single-piece device and the actuator assembly 14 or components providing functionality of the actuator assembly can be fixedly incorporated into the base assembly 12.

Base assembly 12 includes a base 16 having a lower surface 18 that can be positioned on the skin of a patient during use, and an opposite, upper surface 20. One or more indicia such as lines 22 are located on the base 16, and are configured for alignment with external alignment sources such as light beams to facilitate alignment of the marker 10 over an isocenter on the patient's body. In the illustrated embodiment the lines 22 converge or intersect at a location 24 on the base 16 (e.g., in the center of the base) that can be positioned over the desired isocenter location on the patient.

Base assembly 12 includes an ink reservoir 28 and a valve 30. Ink reservoir 28 has an inner surface 32 defining a channel 34 in which the valve 30 is located. Ink is contained within the reservoir 28. An opening 36 through the inner surface 32 provides a flow path for ink from reservoir 28 as described below. Although the reservoir 28 is shown as a cylindrical member in FIG. 1, other embodiments of marker 10 have reservoirs with other shapes. Valve 30 has an inner surface 40 defining an inner or ink channel 42, an outer surface 44, and a channel or opening 46 that extends through the valve between the outer surface and ink channel. Valve 30 is configured for movement with respect to the reservoir 28 between first and second positions. In the first position shown in FIG. 1, the opening 46 is located at a position that is spaced from the opening 36 of the reservoir, and a portion of the outer surface 44 of the valve is located adjacent to the opening in the reservoir, thereby sealing the reservoir and preventing the flow of ink into the ink channel 42. As described in greater detail below, the valve 30 can be rotated or otherwise moved to the second position at which the opening 46 is aligned with the opening 36 of the reservoir 28 to allow ink from the reservoir to flow into the ink channel 42 of the valve. Other embodiments have other valve structures to control the flow ink from the reservoir into an ink channel.

Actuator assembly 14 includes a marker such as needle 50 which has a tip 52 at its distal end, a valve actuator 54 and a marker actuator 56. In embodiments, the needle tip 52 is hollow or has a recess at its distal end to collect ink during actuation of the actuator assembly 14. Valve actuator 54, which can be grasped and manipulated by the user's hand, is configured to engage the valve 30 and enable the rotation of the valve by a user of the marker 10. In the illustrated embodiment the actuator 54 includes an engagement structure such as tabs 53 that extend from the bottom of the actuator. The engagement structure (e.g., tabs 53) are configured to engage the valve 30, and in the illustrated embodiment the valve includes an engagement structure in the form or recesses 58 that receive the tabs 53 when the actuator assembly 14 is mounted to the base assembly 12. Other embodiments of the marker 10 include other structures such as complimentary luer lock elements on one or both of the valve actuator 54 and the valve 30. Yet other embodiments have yet other structures to rotate or otherwise actuate the valve. Actuator assembly 14 is configured to position the needle 50 at the location 24 corresponding to the isocenter location on the patient when mounted to the base assembly 12.

Marker actuator 56 is a plunger type device in the illustrated embodiment, and includes a shaft 60 and a head 62. The needle 50 extends from an end of the shaft 60 opposite the head 62, over the isocenter location 24. A biasing mechanism such as spring 64 biases the marker actuator 56 to a retracted position with the tip 52 of the needle 50 located above the lower surface 18 of the base 16. During use of the marker 10, after the actuator assembly 14 is mounted to the base assembly 12 and the valve actuator 54 is actuated to fill the ink channel 42, a user actuates the actuator 56 to drive the needle tip 52 through the ink-filled ink channel to a marking position in the patient's skin. An isocenter mark can thereby be tattooed onto the patent at the isocenter. When the actuator 56 is released, the spring 64 will return the actuator to the retracted position and withdraw the needle 50 from the patient's skin. The marker 10 can then be removed from the patient.

In embodiments, the end of the ink channel 42 facing the lower surface 18 of the base 16 is open, and configured to enable capillary forces (based e.g., on the ink viscosity and the surface tension between the ink and the surface of the ink channel) to retain the ink within the ink channel when the marker 10 is removed from the patient. In other embodiments the portion of the lower surface 18 of the base 16 includes material (e.g., self-sealing silicone, liquid silicone rubber) that the needle 50 pierces when the actuator 56 is actuated, and that seals the opening sufficiently to prevent or minimize ink flow following retraction of the needle. The marker actuator 56 can be configured to cause the needle 50 to penetrate the patient's skin by the desired amount. In the illustrated embodiment, for example, the actuator assembly 14 is configured so the valve actuator 54 functions as a stop for the head 62 with the needle 50 extending below the base 16 by the desired distance when the actuator 56 is driven to its marking position. In embodiments, the actuator assembly 14 is configured to have the tip 52 of needle 50 located within the valve actuator 54 when the actuator assembly is in the retracted position (i.e., the valve actuator functions as a housing for the needle).

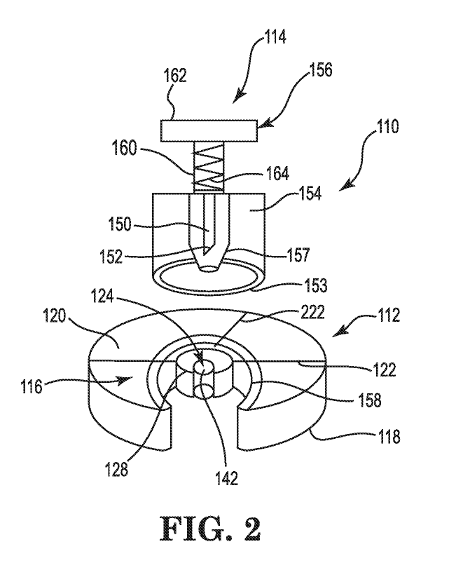

FIG. 2 is a diagrammatic illustration of an isocenter marker 110 in accordance with embodiments of the invention. As shown, marker 110 includes a base assembly 112 and an actuator assembly 114 that can be attached to the base assembly. In the illustrated embodiment the marker 110 is manufactured as a multi-piece device, and the actuator assembly 114 is a separate element that can be mounted to the base assembly 112 during use of the marker 110. In other embodiments the marker 110 is a single-piece device and the actuator assembly 114 or components providing functionality of the actuator assembly can be fixedly incorporated into the base assembly 112.

Base assembly 112 includes a base 116 having a lower surface 118 that can be positioned on the skin of a patient during use, and an opposite, upper surface 120. One or more indicia such as lines 122 are located on the base 116, and are configured for alignment with external alignment sources such as light beams to facilitate alignment of the marker 110 over an isocenter on the patient's body. In the illustrated embodiment the lines 122 converge or intersect at a location 124 on the base 116 (e.g., in the center of the base) that can be positioned over the desired isocenter location on the patient.

Base assembly 112 includes an ink reservoir 128 and an ink channel 142. As described in greater detail below, the ink reservoir 128 is located at a position with respect to the ink channel 142 that allows ink from with the reservoir to flow into the channel when the reservoir is ruptured or otherwise opened by the actuator assembly 114. In the illustrated embodiment the ink reservoir 128 surrounds the ink channel 142. The ink reservoir 128 has other shapes and/or is located at other locations with respect to the ink channel 142 in other embodiments. In embodiments, the ink reservoir 128 can be an element that is separate from the base 116, and positioned on the base (e.g., in a recess in the upper surface of the base) prior to use of the marker 110. Ink reservoir 128 can be formed from materials such as polyimide, self-sealing silicone, liquid silicone rubber or others.

Actuator assembly 114 includes a marker such as needle 150 which has a tip 152 at its distal end, a housing 154, a marker actuator 156 and a rupture member 157. In embodiments, the needle tip 152 is hollow or has a recess at its distal end to collect ink during actuation of the actuator assembly 114. Housing 154, which can be grasped and manipulated by the user's hand, is configured to engage the base 116. In the illustrated embodiment the housing 154 includes an engagement structure such as locking ring 153 on the bottom portion of the housing. The engagement structure (e.g., locking ring 153) is configured to engage the base 116, and in the illustrated embodiment the base includes an engagement structure in the form of locking channel 158 that receives the locking ring when the actuator assembly 114 is mounted to the base assembly 112. Other embodiments of the marker 110 include other structures such as complimentary luer lock elements on one or both of the housing 154 and the base 116 to engage the housing and base. Actuator assembly 114 is configured to position the needle 150 at the location 124 corresponding to the isocenter location on the patient when mounted to the base 116.

Rupture member 157 is configured to engage the ink reservoir 128, and to rupture or otherwise open the ink reservoir, when the actuator assembly 114 is mounted to the base assembly 112. In the illustrated embodiment, for example, the lower portion of the rupture member 157 is tapered and sufficiently sharp to puncture the ink reservoir 128. In other embodiments the rupture member 157 takes other structural forms. In still other embodiments the rupture member can be configured to be separately actuated to rupture the ink reservoir (e.g., engaging the actuator assembly on the base assembly does not cause the rupture of the ink reservoir).

Marker actuator 156 is a plunger type device in the illustrated embodiment, and includes a shaft 160 and a head 162. The needle 150 extends from an end of the shaft 160 opposite the head 162, over the isocenter location 124. A biasing mechanism such as spring 164 biases the marker actuator 156 to a retracted position with the tip 152 of the needle 150 located above the lower surface 118 of the base 116. In the illustrated embodiment the needle tip 152 is within the housing 154 when the marker actuator is in the retracted state. During use of the marker 110, after the actuator assembly 114 is mounted to the base assembly 112 and the rupture member 157 has opened the ink reservoir 128 to fill the ink channel 142, a user actuates the actuator 156 to drive the needle tip 152 through the ink-filled ink channel to a marking position in the patient's skin. An isocenter mark can thereby be tattooed onto the patent at the isocenter. When the actuator 156 is released, the spring 164 will return the actuator to the retracted position and withdraw the needle 150 from the patient's skin. The marker 110 can then be removed from the patient.

In embodiments, the end of the ink channel 142 facing the lower surface 118 of the base 116 is open, and configured to enable capillary forces (based e.g., on the ink viscosity and the surface tension of the ink within the ink channel) to retain the ink within the ink channel when the marker 110 is removed from the patient. In other embodiments, the portion of the lower surface 118 of the base 116 includes material (e.g., self-sealing silicone, liquid silicone rubber) that the needle 150 pierces when the actuator 156 is actuated, and that seals the opening sufficiently to prevent or minimize ink flow following retraction of the needle. The actuator 156 can be configured to cause the needle 150 to penetrate the patient's skin by the desired amount. In the illustrated embodiment, for example, the actuator assembly 114 is configured so the housing 154 functions as a stop for the head 162 with the needle tip 152 extending below the base 116 by the desired distance when the actuator 156 is driven to its marking position.

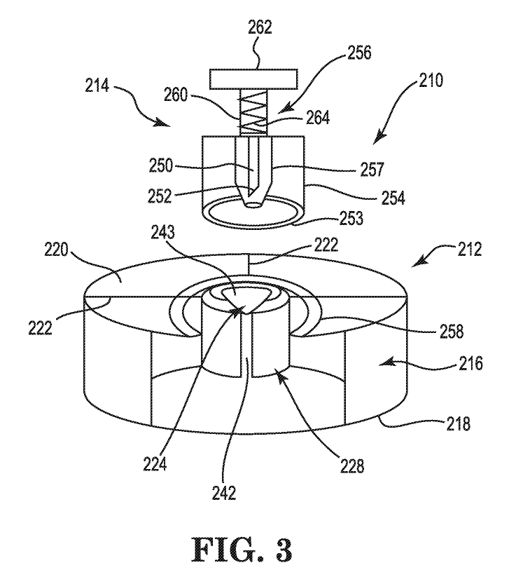

FIG. 3 is a diagrammatic illustration of an isocenter marker 210 in accordance with embodiments of the invention. As shown, marker 210 includes a base assembly 212 and an actuator assembly 214 that can be attached to the base assembly. In the illustrated embodiment the marker 210 is manufactured as a multi-piece device, and the actuator assembly 214 is a separate element that can be mounted to the base assembly 212 during use of the marker 210. In other embodiments the marker 210 is a single-piece device and the actuator assembly 214 or components providing functionality of the actuator assembly can be fixedly incorporated into the base assembly 212. As described below, in yet other embodiments the marker 210 is a three-piece device, having an ink reservoir 228 that can be separately formed an inserted onto other components of the base assembly 212 when the device is used.

Base assembly 212 includes a base 216 having a lower surface 218 that can be positioned on the skin of a patient during use, and an opposite, upper surface 220. One or more indicia such as lines 222 are located on the base 216, and are configured for alignment with external alignment sources such as light beams to facilitate alignment of the marker 210 over an isocenter on the patient's body. In the illustrated embodiment the lines 222 converge or intersect at a location 224 on the base 216 (e.g., in the center of the base) that can be positioned over the desired isocenter location on the patient.

Base assembly 212 includes an ink reservoir 228 having an ink channel 242 and a valve or other openable seal 243 on an upper portion of the ink channel. Ink (not shown) is contained within the ink channel. Seal 243 can include structures such as a flap-type or leaflet-type valves that seal the upper portion of the ink channel 242 when closed, while being capable of being opened to provide an opening into the ink channel. The seal 243 is closed prior to use of the marker 210, and as described below is opened during use of the device. In embodiments, the lower end portion of the ink channel (e.g., the portion adjacent the lower surface 218 of base 216) is open, and ink channel 242 and seal 243 are configured (e.g., the size of the opening and the material of the inner surface) in connection with characteristics of the ink (e.g., its surface tension and viscosity), to enable capillary or other forces to retain the ink within the ink channel while the seal is closed. Ink reservoir 228 can be fabricated as part of the base assembly 212. In other embodiments the ink reservoir 228 is manufactured as a separate element, and can be mounted to the base 216 when the marker 210 is being used.

Actuator assembly 214 includes a marker such as needle 250 which has a tip 252 at its distal end, a housing 254, a marker actuator 256 and a seal actuator 257. In embodiments, the needle tip 252 is hollow or has a recess at its distal end to collect ink during actuation of the actuator assembly 214. Housing 254, which can be grasped and manipulated by the user's hand, is configured to engage the base 216. In the illustrated embodiment the housing 254 includes an engagement structure such as locking ring 253 on the bottom portion of the housing. The engagement structure (e.g., locking ring 253) is configured to engage the base 216, and in the illustrated embodiment the base includes an engagement structure in the form of locking channel 258 that receives the locking ring when the actuator assembly 214 is mounted to the base assembly 212. Other embodiments of the marker 210 include other structures such as complimentary luer lock elements on one or both of the housing 254 and the base 216 to engage the housing and base. Actuator assembly 214 is configured to position the needle 250 at the location 224 corresponding to the isocenter location on the patient when mounted to the base 116.

Seal actuator 257 is configured to engage and open the seal 243 when the actuator assembly 214 is mounted to the base assembly 212. In the illustrated embodiment, for example, the lower portion of the seal actuator 257 is tapered and pushes movable components of the seal 243 (e.g., one or more flaps or leaflets) into the ink channel 242. In other embodiments the seal actuator 257 can be configured to be separately actuated to open the seal 243 (e.g., engaging the actuator assembly on the base assembly does not cause the seal to open).

Marker actuator 256 is a plunger type device in the illustrated embodiment, and includes a shaft 260 and a head 262. The needle 250 extends from an end of the shaft 260 opposite the head 262, over the isocenter location 224. A biasing mechanism such as spring 264 biases the marker actuator 256 to a retracted position with the tip 252 of the needle 250 located above the lower surface 218 of the base 216. In the illustrated embodiment the needle tip 252 is within the housing 254 when the marker actuator is in the retracted state. During use of the marker 210, after the actuator assembly 214 is mounted to the base assembly 212 and the seal actuator 257 has opened the seal 243, a user actuates the actuator 256 to drive the needle tip 252 through the ink-filled ink channel 242 to a marking position in the patient's skin. An isocenter mark can thereby be tattooed onto the patent at the isocenter. When the actuator 256 is released, the spring 264 will return the actuator to the retracted position and withdraw the needle 250 from the patient's skin. The marker 210 can then be removed from the patient. In embodiments, the actuator assembly 214 can be removed from the base assembly 212 before the marker is removed from the patient, allowing a biasing force on the seal 243 to return the seal to its closed position, and thereby prevent or minimize ink flow following retraction of the needle. The actuator 256 can be configured to cause the needle 250 to penetrate the patient's skin by the desired amount. In the illustrated embodiment, for example, the actuator assembly 214 is configured so the housing 254 functions as a stop for the head 262 with the needle tip 252 extending below the base 216 by the desired distance when the actuator 256 is driven to its marking position.

Although the invention has been described with reference to preferred embodiments, those of skill in the art will recognize that changes can be made in form and detail without departing from the spirit and scope of the invention.

1. An isocenter marker, comprising:
a base;
alignment indicia configured for alignment with external alignment sources to facilitate alignment of the base over an isocenter on a patient's body;
an ink reservoir on the base, including:
an inner surface defining a channel; and
an opening between the reservoir and the channel;
a valve in the channel of the ink reservoir, including:
an outer surface;
an inner surface defining an inner channel;
an opening between the outer and inner surfaces; and
wherein the valve is rotatable between a first position at which the opening of the valve is aligned with the inner surface of the ink reservoir to prevent ink flow from the reservoir to the inner channel of the valve, and a second position at which the opening of the valve is aligned with the opening of the ink reservoir to allow ink flow from the reservoir to the inner channel of the valve;
an ink marker including a needle with a tip configured for movement through the inner channel of the valve and positioned relative to the alignment indicia for marking an ink mark indicating the isocenter;
a valve actuator for rotating the valve between the first and second positions; and
a marker actuator for actuating the ink marker to drive the needle from a retracted position through the inner channel of the valve to a marking position.

2. The isocenter marker of claim 1 wherein the alignment indicia are on the base.

3. The isocenter marker of claim 1 wherein the channel of the ink reservoir is a cylindrical channel.

4. The isocenter marker of claim 1 wherein the valve actuator and marker actuator comprise an assembly.

5. The isocenter marker of claim 4 wherein the valve actuator and marker actuator assembly is removably attachable to the base and/or ink reservoir.

6. The isocenter marker of claim 5 wherein the valve actuator includes an engagement structure to engage the valve, and wherein rotation of the valve actuator rotates the valve.

7. The isocenter marker of claim 1 wherein the marker actuator includes a biasing mechanism to bias the ink marker toward the retracted position.

8. The isocenter marker of claim 1 wherein a patient-engaging portion of the ink reservoir channel is open, and the needle is driven through the open patient-engaging portion of the ink reservoir in response to actuation of the marker actuator.

9. The isocenter marker of claim 1 wherein a patient-engaging portion of the ink reservoir includes a puncturable barrier to retain ink in the channel of the reservoir, and the needle is driven through the barrier in response to actuation of the marker actuator.

10. An isocenter marker, comprising:
a base;
alignment indicia configured for alignment with external alignment sources to facilitate alignment of the base over an isocenter on a patient's body;
an ink reservoir in the base;
an ink channel in the base;
a valve coupling the ink reservoir to the ink channel, wherein the valve is movable between a first state to prevent ink flow from the reservoir to the ink channel, and a second state to allow ink flow from the reservoir to the ink channel;
an ink marker including a needle with a tip configured for movement through the ink channel and positioned relative to the alignment indicia for marking an ink mark indicating the isocenter;
a valve actuator for moving the valve between the first and second states; and
a marker actuator for actuating the ink marker to drive the needle from a retracted position through the ink channel to a marking position.

11. The isocenter marker of claim 10 wherein the actuator assembly is removably attachable to the base and includes an engagement structure for engaging the base.

12. The isocenter marker of claim 10 wherein the ink reservoir is attachable to the base.

13. The isocenter marker of claim 10 wherein the valve actuator and marker actuator comprise an assembly attachable to the base.

14. An isocenter marker, comprising:
a base;
alignment indicia configured for alignment with external alignment sources to facilitate alignment of the base over an isocenter on a patient's body;
an ink reservoir in the base;
an ink channel in the base;
an actuator assembly attachable to the base, including:
a rupture member to open the ink reservoir and allow ink from the reservoir to flow to the ink channel when the actuator assembly is attached to the base;
an ink marker including a needle with a tip configured for movement through the ink channel and positioned relative to the alignment indicia for marking an ink mark indicating the isocenter; and
a marker actuator for actuating the ink marker to drive the needle from a retracted position through the ink channel to a marking position.

15. The isocenter marker of claim 14 wherein the ink reservoir is a ring-shaped member extending around the ink channel.

16. The isocenter marker of claim 14 wherein the rupture member includes a tapered end portion that extends into the ink channel and engages the ink reservoir when the actuator assembly is attached to the base.

17. The isocenter marker of claim 14 wherein the actuator assembly is removably attachable to the base.

18. An isocenter marker, comprising:
a base;
alignment indicia configured for alignment with external alignment sources to facilitate alignment of the base over an isocenter on a patient's body;
an ink channel through the base, the ink channel having upper and lower end portions;
an openable seal over the upper end portion of the ink channel;
a seal actuator attachable to the base to open the seal;
an ink marker including a needle with a tip configured for movement through the open seal and ink channel and positioned relative to the alignment indicia for marking an ink mark indicating the isocenter; and
a marker actuator for actuating the ink marker to drive the needle from a retracted position through the open seal and ink channel to a marking position.

19. The isocenter marker of claim 18 wherein the seal actuator, ink marker and marker actuator comprise an assembly that is attachable to the base.

20. The isocenter marker of claim 19 wherein the assembly is removably attachable to the base.

21. The isocenter marker of claim 18 wherein the ink channel and seal comprise an assembly attachable to the base.
